(19) United States
(12) Patent Application Publication
(10) Pub. No.: US 2014/0242110 A1
(43) Pub. Date: Aug. 28, 2014

(54) Dose, localization, and formulation of botulinum toxins in skin and muscle

(71) Applicant: DT Scimed, LLC, Towson, MD (US)

(72) Inventor: L. Andrew Koman, Winston-Salem, NC (US)

(21) Appl. No.: 13/815,399

(22) Filed: Feb. 28, 2013

(51) Int. Cl. A61K 39/385

(52) U.S. Cl. CPC A61K 39/385
USPC 424/197.11

(57) ABSTRACT

Formulations of and dosing protocols for the administration of botulinum toxin that maximize efficacy and specificity while minimizing the likelihood of overdosing and undesirable side effects of treatment. The formulations include positively charged carriers, such as cationic peptides, which otherwise have no inherent botulinum-toxin-like activity. The dosing regimen is based on the pattern, quantity, and location of neuromuscular junctions in the target tissue. Because the number of neuromuscular junctions in a target tissue remains generally stable throughout life and because the pharmacological effect of botulinum toxin is localized at the neuromuscular junction, dosing efficacy is unaffected by muscle mass, age of the patient, or body weight.

### RELATED APPLICATIONS

This is a Continuation-In-Part of pending patent application Ser. No. 12/588,345 filed Oct. 13, 2009 which claims priority from U.S. Provisional Application No. 61/136,908, filed Oct. 14, 2008.

### STATEMENT REGARDING FEDERALLY SPONSORED RESEARCH OR DEVELOPMENT

None

### REFERENCE TO SEQUENCE LISTING, A TABLE OR A COMPUTER PROGRAM LISTING COMPACT DISC APPENDIX

None.

### BACKGROUND OF THE INVENTION

1. Field of Invention

This invention relates to formulations of and dosing protocols for the administration of botulinum toxin that maximizes efficacy and specificity while minimizing the likelihood of overdosing and undesirable side effects of treatment.

2. Background of the Invention

Botulinum toxins, in particular botulinum toxin type A, have been used in the treatment of a number of neuromuscular disorders and conditions involving muscular spasticity as well as in cosmetic procedures; for example, strabismus, blepharospasm, spasmodic torticollis (cervical dystonia), oromandibular dystonia and spasmodic dysphonia (laryngeal dystonia). The toxin binds rapidly and strongly to presynaptic cholinergic nerve terminals and inhibits the exocytosis of acetylcholine, decreasing acetylcholine release, thereby reducing or eliminating the activation of postsynaptic muscles, nerves, or effector tissues. This results in local paralysis and hence relaxation of the muscle afflicted by spasm.

The term botulinum toxin as used herein is a generic term embracing the family of toxins produced by the anaerobic bacterium Clostridium botulinum and, to date, seven immunologically distinct toxins have been identified. These have been given the designations A, B, C, D, E, F and G. For further information concerning the properties of the various botulinum toxins, reference is made to the article by Jankovic & Brin, The New England Journal of Medicine, pp 1186-1194, No 17, 1991 and to the review by Charles L Hatheway, Chapter 1 of the book entitled Botulinum Neurotoxin and Tetanus Toxin Ed. L. L. Simpson, published by Academic Press Inc. of San Diego Calif. 1989, the disclosures in which are incorporated herein by reference.

The neurotoxic component of botulinum toxin has a molecular weight of about 150 kilodaltons and is believed to comprise a short polypeptide chain of about 50 kD, which is considered to be responsible for the toxic properties of the toxin, and a larger polypeptide chain of about 100 kD which is believed to be necessary to enable the toxin to penetrate the nerve. These so-called “short” and “long” chains are linked together by disulphide bridges.

Intramuscular injections of botulinum toxin A are generally used to balance muscle forces across joints, to diminish or decrease painful spasticity, to decrease deforming forces through selective motor paralysis, to diminish neuropathic and nociceptive pain, to diminish dystonic contractures, to decrease muscle deformation after injury or surgery, and to diminish sweating. The target organelles contain soluble NSF attachment receptor (SNARE) proteins and neurotransmitter-containing vesicles which require these SNARE proteins for fusion of the vesicle to the cell membrane and release of neurotransmitter. Targets include neuromuscular junctions, sweat glands, vascular beds and nociceptors.

Therapeutic use of these toxins represents a somewhat unique pharmacokinetic profile. In order for toxin to produce its desired action, it must not only be delivered to the target tissue, e.g. muscle (usually by direct injection), but it must also bind to terminal portions of nerves innervating the target tissue (i.e. the neuromuscular junction), and be transported across the presynaptic terminal membrane into the intracellular domain where the active molecule is cleaved from the binding portion of the divalent complex. Then the active molecule must bind irreversibly and enzymatically inactivate molecules in the nerve terminal specific for neurochemical transmission. Thus the toxin molecules are not delivered systemically to distribute throughout the body. The ultimate target is not a specific muscle or organ but rather molecules located in specific nerves which innervate the target tissue within an anatomically defined region of the target tissue or muscle. For example, within skeletal muscle fibers, nerves do not uniformly distribute through the muscle but rather the terminals of the nerves are restricted to a certain region of the muscle. In the case of muscle fibers, prior research has shown that different muscles have different numbers of neuromuscular junctions and the total number of these neuromuscular junctions is not dependent on the mass or volume of the muscle or the individual but rather on other factors such as the function of the muscle fibers.

Current recommendations and dosing regimens are empirical and utilize dosage based upon bodyweight in, for example, the management of cerebral palsy and in orthopaedic uses. With specific regard to its use in children, the use of botulinum toxin in the management of cerebral palsy and in orthopaedic usage is based on the size and weight of the growing child, rather than age, to insure safety since overall toxicity data was based upon units per kilogram of body weight in primates. U.S. Pat. No. 6,395,277 issued 28 May 2002 shows a dosing regimen for the treatment of cerebral palsy, noting that dosing should occur “preferably . . . in the region of the neuromuscular junction” according to “the number of muscle groups requiring treatment, the age and size of the patient.” Similar dosing regimens base relative dosages upon the size of muscle.

Historically, dosage recommendations for the administration of botulinum toxin have been an imprecise science. Recommendations have been made on the basis of body weight, body surface area, size or volume occupied by a specific muscle, etc. The overreaching goal for each of these therapeutic or cosmetic uses of botulinum toxins is that the toxin be administered in a dosage and volume appropriate to achieving the desired response while remaining localized within the desired specific region of injection. Because the ultimate site of toxin action are nerve junctions within certain regions of the target tissue, over- and under-dosing remains a significant challenge. Administration of too high an absolute dose (total number of toxin molecules relative to the total number of neuromuscular junction targets) or too high a volume of injection might produce adverse reactions related to diffusion of the toxin. Diffusion of the toxin into undesired areas can produce inappropriate paralysis or pathophysiological responses. Too high a dose will produce the desired effect of tissue paralysis but also result in toxin distribution to non targeted tissues thereby causing an unintended loss of physiological function in those tissue areas. Additionally, delivery of supraoptimal toxin doses presents an undesired immunological challenge which may cause reduced effectiveness on subsequent administrations of the toxin. Using currently available formulations of toxin and previously known dosing regimens, when a large volume of toxin is delivered, it is likely that toxin molecules will diffuse to distant targets resulting in the dilution of the effect of the toxin at the desired target and inappropriately exposing other regions to the toxin. In a large volume dosing scenario, a higher overall dose of toxin would be required at a later time to overcome the dilution effect thus increasing the exposure of other tissues. In these cases where inappropriate doses or volumes are used, not only may patient safety be compromised but the cost of the procedure is increased due to wasted toxin or treatment of unanticipated pharmacological outcomes.

Perhaps the most obvious examples of this inappropriate dosage are delivery of toxin based on body weight to individuals who are at the extremes of weight distribution curves. The toxin acts at the neuromuscular junction and the quantity of the aforementioned junctions does not change proportionately with changes in body mass. Hence, in these cases, individuals with high and low body mass would receive inappropriately high or low doses, respectively.

Various recommendations have demonstrated clinical usefulness but fail to address that 1) the toxin acts at the neuromuscular junction, and 2) the number of neuromuscular junctions varies from muscle to muscle, and 3) the number of neuromuscular junctions tends not to vary as a person ages. Neuromuscular junctions for individual muscles are not directly proportional to muscle mass or volume. Rather, the distribution of neuromuscular junctions varies from muscle to muscle and the number of neuromuscular junctions is affected minimally by age and total body weight. The existing dosage recommendations are clinically efficacious in 50 to 70 percent of patients, namely large toddlers and adolescents, but may underdose infants and small toddlers and overdose heavy children, teenagers, and adults. What is needed are more precise dosing methods to delineate optimal number of units, volume, and injection sites for individual muscles. Preferably, such regimens would, in addition, utilize formulations of toxin that are less likely to diffuse from the immediate areas of injection, thereby improving efficacy, minimizing protein antigen load and subsequent antibody formation, and decreasing costs.

### SUMMARY OF THE INVENTION

The present invention discloses novel dosing methods and formulations for botulinum toxin. These methods are based on the number and distribution of neuromuscular junctions in the target muscle and include determining the mass of the target muscle, determining the distribution and location of neuromuscular junctions in that muscle, and injecting an appropriate therapeutic dose of botulinum toxin in the vicinity of and according to the quantity of neuromuscular junctions in the muscle. The botulinum toxin formulations include non-native, non-covalently associated molecules having a positively charged backbone which minimizes diffusion from the injection site. Dosing regimens are based on the quantity of neuromuscular junctions in the aforementioned tissue and utilize toxin formulations that discourage excess diffusion from the injection site ensure efficacy, while minimizing possible side effects and minimizing cost by ensuring that only that amount of toxin necessary to achieve the desired effect is used.

It is an object of this invention to provide novel formulations for botulinum toxins;

It is further an object of this invention to provide formulation for botulinum toxin that minimize diffusion from the injection site;

It is another object of this invention to provide a safe dosing method for botulinum toxins;

It is yet another object of this invention to provide an efficacious method for dosing botulinum toxins;

It is still another object of this invention to provide a minimally invasive means of dosing botulinum toxins;

It is yet another object of this invention to provide a cost effective dosing method for botulinum toxins; and,

It is an object of this invention to provide a simple, easily complied with dosing method for the use of botulinum toxins.

### BRIEF DESCRIPTION OF THE DRAWINGS

FIG. 1 shows a rat gastrocnemius muscle;

FIG. 2 graphs the dose response recovery of botulinum toxin at dosages of 1 U/kg, 3 U/kg and 6 U/kg;

FIG. 3 graphs the effect of different volumes of toxin injection;

FIG. 4 shows a unipennate muscle with a single transverse band of neuromuscular junctions;

FIG. 5 shows a unipennate gracilis muscle with two transverse bands of neuromuscular junctions;

FIG. 6 shows a bipennate converging biceps brachii muscle having neuromuscular junctions located in an inverted “U” shape;

FIG. 7 shows a rectus femoris muscle with two bands of neuromuscular junctions running along its length; and,

FIG. 8 shows a deltoid muscle with an irregular pattern of neuromuscular junctions.

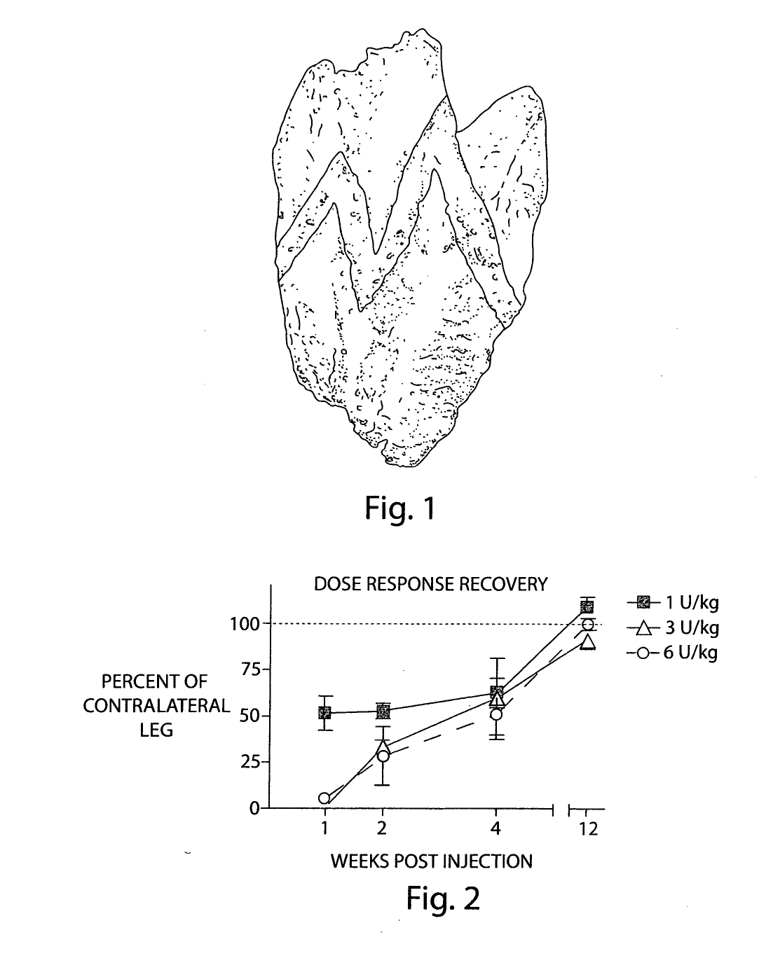

These and other objects, advantages, and novel features of the present invention will become apparent when considered with the teachings contained in the detailed disclosure along with the accompanying drawings.

### DESCRIPTION OF THE INVENTION

While the invention is described in connection with certain preferred embodiments, it is not intended that the present invention be so limited. On the contrary, it is intended to cover all alternatives, modifications, and equivalent arrangements as may be included within the spirit and scope of the invention as defined by the appended claims.

The invention discloses novel dosing methods for botulinum toxin based on the quantity and distribution of neuromuscular junctions in a target tissue. In a preferred embodiment, the invention further utilizes novel formulations of toxin that minimize diffusion from the site of injection to thereby maximize efficacy. Previous recommendations for dosing were based on, for example, body mass and/or age. In the present invention, therapeutic dosing is based on 1) the quantity and distribution of neuromuscular junctions and 2) the volume of liquid or other carrier material in which that dose is delivered to the target tissue. In a preferred embodiment, novel formulations of toxin which minimize diffusion from the injection site are used in lieu of previously known formulations. This results in decreased incidences of under or over-dosing, minimized direct costs of administrating the substance due to the more efficient use of the toxin itself, and minimized indirect costs resulting from the medical costs avoided by eliminating the likelihood of anaphylaxis and immuno-challenge resulting from too high a dose. Ideal dose in units is therefore based upon the number of NSF attachment receptor (SNARE) containing organelles (i.e., neuromuscular junctions to be blocked); the volume or concentration calculated from the muscle mass; the number of injection sites is dictated by the length and width of the target tissue; and injection of novel formulations of toxin which minimize diffusion from the injection site.

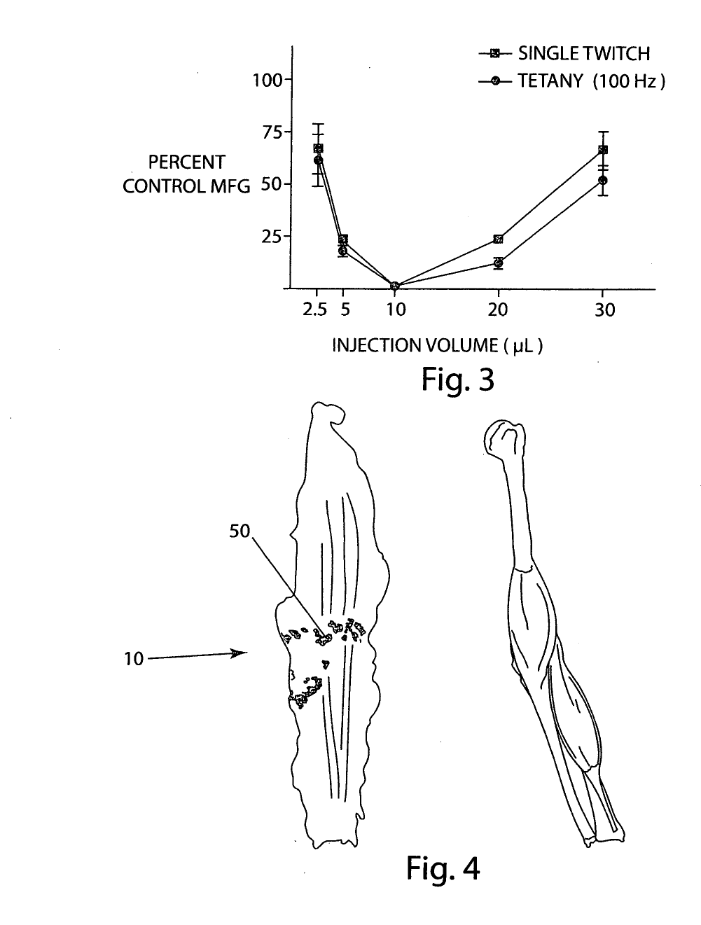

As shown in the Dose Response Recovery Graph of FIG. 2, because its efficacy is dependant on the quantity of neuromuscular junctions in the target tissue, dosing of botulinum toxin exhibits clear maximum dosing behavior beyond which an increased dose shows no appreciable change in effect. The neuromuscular junctions of the target tissue are saturated such that additional availability of toxin produces no additional effect. The graph of FIG. 3, however, shows clearly that proper titration of the dose is important. While suboptimal amounts of toxin obviously produce lower degrees of relaxation, supraoptimal doses produce similarly reduced results. It is believed that the reduced results occur because toxin molecules diffuse away from the target site resulting in the dilution of the effect of the toxin at the desired target and inappropriately expose other regions to the toxin. Hence determination of the most efficacious dosage for a target muscle is critical and in a preferred embodiment will include utilization of novel toxin formulations to minimize diffusion.

Botulinum toxin A is produced by Allergan Pharmaceuticals as BOTOX® and by Ipsen Pharmaceuticals, Ltd. as DYSPORT®. Each vial of BOTOX® contains 100 units of C. botulinum type A neurotoxin complex, 0.5 mg of human albumin, and 0.9 mg of sodium chloride as a vacuum-dried frozen powder that requires reconstitution. One unit of BOTOX® is equal to the median intraperitoneal lethal dose (LD50) in Swiss-Webster mice weighing 18 to 20 g. The LD50 for Botox® has been calculated in primates at 39 to 56 units/kg body wt. However, the exact lethal dose in humans is unknown. The calculated human LD50 of 59 units is based on an extrapolation of data. DYSPORT® clostridium botulinum type A toxin-hemogglutinin complex is available in 500-unit vials. DYSPORT® units of activity equal 1 mouse LD50 based on their specific assay technique and is sometimes referred to in nanograms, with 1 nanogram equal to 40 units. In the United Kingdom and many other countries, it is approved and labeled for multiple indications, including spasticity of the arm in patients following stroke, dynamic equinus foot deformity due to spasticity in ambulant pediatric cerebral palsy patients, two years of age or older, spasmodic torticollis, blephorospasm, and hemifacial spasm. With regard to cerebral palsy, DYSPORT® dosing is recommended as “30 units/kg body weight divided between both calf muscles.”

Existing clinical data supports that BOTOX® and DYSPORT® potencies are different; one BOTOX® unit is equal to 2 to 4 DYSPORT® units. Units are not interchangeable between companies or toxin types using package guidelines and suggested dilution tables.

Both BOTOX® and DYSPORT® are reconstituted in injectable physiologic saline prior to intramuscular injection. Both the volume of fluid and number of units of drug must be considered when preparing the toxin for injection. Dosage is defined in absolute terms, based on the number of units per target muscle diluted to volume based on the size of the structure to be injected and quantity and distribution of neuromuscular junctions. The number of units to be injected is calculated by the quantity of neuromuscular junctions to be neutralized, and the volume is determined by the mass of the target muscle, and the number of injection sites by the anatomic distribution of the neuromuscular junctions. Once the appropriate number of active toxin molecules (units) for a given muscle is determined, the dose in units remains constant and the volume and number of injection sites is adjusted based upon growth and anatomy. For example, there are an estimated pikamole of active toxin molecules in 100 units of BOTOX® and an estimated 250,000 neuromuscular junctions in the human biceps brachii. Hence, there are sufficient active toxin molecules to block effectively all neuromuscular junctions of the “target” muscle. The toxin is thereafter injected within the muscle or skin as close to the neuromuscular junctions (or other SNARE-containing organelle) as possible using ultrasonography to localize their position.

In a preferred embodiment a novel formulation of toxin is utilized in lieu of currently available formulations. This formulation is characterized by a reduced tendency to undergo unwanted localized diffusion from the injection site and into surrounding tissues. It has been recognized that certain non-native molecules (i.e., molecules not found in botulinum toxin such as complexes obtained from Clostridium botulinum bacteria) can be added to botulinum toxin, botulinum toxin complexes, and in particular reduced botulinum toxin complexes (as defined herein), to greatly reduce or essentially eliminate toxin diffusion through tissues. The non-native molecules associate non-covalently with the toxin and act as penetration enhancers that improve the ability of the toxin to reach target structures and limit diffusion at the injection site. For example, in certain embodiments, the sample comprising a botulinum toxin may be subjected to clarifying centrifugation and/or filtration to remove gross elements such as whole and lysed cells and cell debris, resulting in a measurably clearer sample. The centrifugation is performed at about 10,000×g to about 30,000×g, most preferably at about 17,700×g. Clarifying filtration will typically comprise normal flow filtration, also called “dead end” filtration, where fluid is moved directly toward a filter media under applied pressure, and particulates too large to pass through the filter pores accumulate at the surface or within the media itself, while smaller molecules pass through as the filtrate. Alternatively, the sample is mixed with ammonium sulfate and normal flow filtration is performed using a filter with a pore size of about 0.1 μm to about 0.3 μm, and more preferably a pore size of about 0.2 μm. In certain particularly preferred embodiments, one or more clarifying steps follow the nuclease digestion step. In yet more preferred embodiments, one or more clarifying steps immediately precede purification by chromatography. Dissociation of the botulinum toxin complex to produce the non-complexed botulinum toxin protein will be understood by one skilled in the art and may be achieved in a number of ways. For example, dissociation may be achieved by raising the pH to about 7.0, or in embodiments in which animal protein free purification is not necessary, treating the complex with red blood cells at a pH of about 7.3. By way of example, the penetration enhancers may be positively charged carriers, such as cationic peptides, which have no inherent botulinum-toxin-like activity and which also contain one or more protein transduction domains as described herein. Such positively charged carrier molecules having protein transduction domains or efficiency groups as described herein, have been found suitable as a transport system for botulinum toxins, enabling toxin to be injected with improved penetration to target structures such as muscles and/or other skin-associated structures, and limiting diffusion into unwanted tissues. The transport occurs without covalent modification of the botulinum toxin. Specifically, “positively charged” and “cationic” as used herein, means that the group in question contains functionalities that are charged under all pH conditions, for instance, a quaternary amine, or contains a functionality which can acquire positive charge under certain solution-phase conditions, such as pH changes in the case of primary amines. More preferably, “positively charged” or “cationic” as used herein refers to those groups that have the behavior of associating with anions over physiologically compatible conditions. One skilled in the art will understand that polymers with a multiplicity of positively-charged moieties need not be homopolymers. Other examples of positively charged moieties are well known and are readily employed. Generally, the positively-charged carrier (also referred to as a “positively charged backbone”) is typically a chain of atoms, either with groups in the chain carrying a positive charge at physiological pH, or with groups carrying a positive charge attached to side chains extending from the backbone. In certain preferred embodiments, the positively charged backbone is a cationic peptide. The term “peptide” may also encompass polypeptides and proteins. In certain preferred embodiments, the positively charged backbone itself will not have a defined enzymatic or therapeutic biologic activity. In certain embodiments, the backbone is a linear hydrocarbon backbone which is, in some embodiments, interrupted by heteroatoms selected from nitrogen, oxygen, sulfur, silicon and phosphorus. The majority of backbone chain atoms are usually carbon. Additionally, the backbone will often be a polymer of repeating units (e.g., amino acids, poly(ethyleneoxy), poly(propyleneamine), polyalkyleneimine, and the like) but can be a heteropolymer. The positively charged backbone may be a polypeptide having protein transduction domains (also referred to as efficiency groups). An efficiency group or protein transduction domain is any agent that has the effect of promoting the translocation of the positively charged backbone through a tissue or cell membrane.

A variety of backbones can be used employing, for example, steric or electronic mimics of polypeptides wherein the amide linkages of the peptide are replaced with surrogates such as ester linkages, thioamides (—CSNH—), reversed thioamide (—NHCS—), aminomethylene (—NHCH₂—) or the reversed methyleneamino (—CH₂NH—) groups, phosphinate (—PO₂RCH₂—), phosphonamidate and phosphonamidate ester (—PO₂RNH—), keto-methylene (—COCH₂—) groups, reverse peptide (—NHCO—), fluoroalkene (—CF═CH—), trans-alkene (—CR═CH—), dimethylene (—CH₂CH₂—), thioether (—CH₂S—), hydroxyethylene (—CH(OH)CH₂—), methyleneoxy (—CH₂O—), tetrazole (CN₄), sulfonamide (—SO₂NH—), methylenesulfonamido (—CHRSO₂NH—), reversed sulfonamide (—NHSO₂—) and backbones with malonate and/or gem-diamino-alkyl subunits. Many of these substitutions result in approximately isosteric polymer backbones relative to backbones formed from α-amino acids. Preferably the positively charged carrier includes side-chain positively charged protein transduction domains in an amount of at least about 0.01%, as a percentage of the total carrier weight, preferably from about 0.01% to about 5%, more preferably from about 0.05% to about 45%, and most preferably from about 0.1% to about 30%. For positively charged protein transduction domains having the formula -(gly)ₙ₁-(arg)ₙ₂-, a preferred range is from about 0.1% to about 25%.

The concentration of positively charged carriers in the compositions according to the invention is sufficient to enhance the delivery of the botulinum toxin to molecular targets such as, for example, motor nerve plates. It is believed that the penetration rate follows receptor-mediated kinetics, such that tissue penetration increases with increasing amounts of penetration-enhancing-molecules up to a saturation point, upon which the transport rate becomes constant. Thus, in a preferred embodiment, the amount of added penetration-enhancing-molecules is equal to the amount that maximizes penetration rate right before saturation. A useful concentration range for the positively charged carrier in the injectable compositions of this invention is about 0.1 pg to about 1.0 mg per unit of the botulinum toxin composition as described herein. More preferably, the positively charged carrier in the topical compositions of the invention is present in the range of about 1.0 pg to 0.5 mg per unit of botulinum toxin.

While botulinum toxin has found use as a therapeutic agent for treating a variety of conditions, it is also the most potent naturally occurring toxin known to humans. Hence, improper administration of the toxin can be extremely dangerous. For instance, accidental systemic delivery of botulinum toxin can lead to paralysis, difficulty breathing, and even death. Moreover, even if botulinum toxin were properly delivered to a localized region of the body as a part of a therapeutic treatment, the toxin has a natural tendency to diffuse over time, thereby increasing the risk of unwanted paralysis in other parts of the body. For example, when botulinum toxin is injected around the eyes to treat wrinkles, it may diffuse to the muscles that control the movement of the eyelids. If this happens, the eyelid muscles may become partially paralyzed, leading to a well known condition know as “eyelid droop,” in which the eyelid is partially closed and interferes with normal vision. Hence, one aspect of this invention is to provide injectable botulinum toxin formulations with an improved safety profile. In preferred embodiments, the injectable botulinum toxin formulations have a reduced tendency to diffuse after injection. Accordingly, preferred formulations of the invention permit more accurate delivery of botulinum toxin and dramatically reduce unwanted side effects associated with uncontrolled local diffusion of toxin.

Table 1 compares the diffusion index profile of an exemplary formulation according to this invention with that of BOTOX®

TABLE 1
Table 1
Botulinum toxin diffusion index measurements for
exemplary formulation, and BOTOX ®
Days Post Treatment

| | 0 | 1 | 2 | 3 | 4 |
|---|---|---|---|---|---|
| Example | N/A | 0 | 0 | 0 | 0 |
| Formula | | | | | |
| BOTOX ® | N/A | 42 | 38 | 38 | 9 |

A comparison of the local diffusion rate following midline injection and lateral-to-midline injection can be made by considering a parameter called the “diffusion index”, which is defined according to the equation:

$$diffusion rate = \frac{midline digital abduction score}{lateral \text{-} to \text{-} medial digital abduction score} \times 100$$

Since digital abduction scores can range from 0 to 4, and lateral-to-midline digital abduction scores are expected to be higher than midline digital abduction scores, diffusion index values will typically range from 0 to 100. A diffusion index value that approaches 100 indicates that the ratio of the midline and lateral-to-midline digital abduction scores approaches unity. This may occur if the rates of diffusion following injection are sufficiently high that the diffusion times for the botulinum toxin to reach and to paralyze the hind digits of the test animal following midline and lateral-to-midline injection are comparable or nearly the same. At the other extreme, diffusion index values that approach zero indicate that the ratio of the midline and lateralto-midline digital abduction scores is approaching zero. This may occur if diffusion of the botulinum toxin following midline injection is so low that it is insufficient to cause paralysis in the hind digits of the test animals, even though paralysis is observed following lateral-to-midline injection.

An exemplary study involved two botulinum toxin formulations, a formulation according to the present invention comprising a buffered and stabilized solution containing the 150 kD type A botulinum toxin molecule non-covalently associated with a positively charged carrier having the formula RKKRRQRRRG-(K)₁₅-SGRKKRRQRRR and BOTOX®. As shown in Table 1, the diffusion index values for the exemplary formulation of the present invention are all zero on the four-day timescale of the experiment, indicating that no paralysis-inducing diffusion is observed following midline injection. This formulation, which contains the type A botulinum toxin molecule non-covalently associated with a positively charged carrier, permits enhanced localization of injected type A botulinum toxin. In this way, the formulation affords an improved safety profile compared to that of BOTOX® and minimizes unwanted paralysis.

Visualization of extremity and trunk muscles is performed reliably using linear probe ultrasonography with a frequency of 5-12 Mhz. For injection localization, linear beam applications better define and delineate the anatomic relationships between muscles, tendons or bones. Higher frequencies are recommended for the localization of the superficial muscles or layers, while lower frequencies may be used for deep structures. The muscles are covered by the epimysium which is the connective tissue that surrounds the entire muscle. The epimysium extends into the muscle to become the perimysium, which divides the fascicle into muscle fibers. The perimysium and the muscular fascicles can be identified because the muscular bundles are hypoechoic (less bright) while the epimysium and perimysium appear as hyperechoic structures. On longitudinal scanning, the fascia is depicted as a fibrillar hyperechoic sheath surrounding the muscle.

There are approximately 250,000 neuromuscular junction in the human biceps brachii muscle. Other human extremity muscles (e.g., the lateral and medial head of the gastrocnemius) have a similar neuromuscular junction density. The total dosage of botulinum toxin (i.e., the absolute number of toxin molecules administered) is given based on the mass of the muscle rather than on the body weight of the individual and injected within 3.0 cm of the area of the muscle containing the neuromuscular junctions (based on ultrasound localization). Thus, for muscles like the soleus where junctions are distributed along the length of muscle fibers, toxin is delivered in multiple locations following the full length of the muscle. In contrast, for muscles like the biceps brachii or medial and lateral head of the gastrocnemius the injection pattern is an inverted U shape following the distribution of the neuromuscular junctions. For example, where 75U is sufficient to produce blockade of the neuromuscular junctions in the lateral gastrocnemius, the biceps brachii has a mass 22% larger than the lateral gastrocnemius, therefore requiring 91U for efficacy. These absolute amounts are then diluted relative to increasing mass and injected adjacent the neuromuscular junctions in the target muscle. Table 2 shows muscles relative to the lateral gastrocnemius and recommended dosages.

TABLE 2

| | | Range of Body wt. (kg) | | | |
|---|---|---|---|---|---|
| | | 0-5 | 5-10 | 10-20 | 20-40 |
| | | Concentration (U/ml) | | | |
| | | 100 | 50 | 25 | 12.5 |
| | | Total U of Botox | | | |
| | mass relative | 75 | 75 | 75 | 75 |
| | to lateral | ml | 2X ml | 3X ml | 4-5 X ml |
| | gastroc- | per | per | per | per |
| muscle | nemius m. | muscle | muscle | muscle | muscle |
| lateral | 1.00 | 0.8 | 1.5 | 2.3 | 3.4 |
| gastrocnemius | | | | | |
| medial gastroc | 1.48 | 1.1 | 2.2 | 3.3 | 5.0 |
| tibialis | 0.84 | 0.6 | 1.3 | 1.9 | 2.8 |
| posterior | | | | | |
| tibialis | 0.66 | 0.5 | 1.0 | 1.5 | 2.2 |
| anterior | | | | | |
| soleus | 2.63 | 2.0 | 3.9 | 5.9 | 8.9 |
| FHL | 0.44 | 0.3 | 0.7 | 1.0 | 1.5 |
| Sartorius | 0.56 | 0.4 | 0.8 | 1.3 | 1.9 |
| Semi- | 0.98 | 0.7 | 1.5 | 2.2 | 3.3 |
| membranosus | | | | | |
| Semi- | 0.70 | 0.5 | 1.1 | 1.6 | 2.4 |
| tendinosus | | | | | |
| Gracilis | 0.32 | 0.2 | 0.5 | 0.7 | 1.1 |
| Pronator Teres | 0.23 | 0.2 | 0.3 | 0.5 | 0.8 |
| Biceps | 1.22 | 0.9 | 1.8 | 2.7 | 4.1 |
| Brachioradialis | 0.39 | 0.3 | 0.6 | 0.9 | 1.3 |
| Pronator | 0.07 | 0.1 | 0.1 | 0.2 | 0.2 |
| Quadratus | | | | | |
| Supinator | 0.21 | 0.2 | 0.3 | 0.5 | 0.7 |
| FCU | 0.14 | 0.1 | 0.2 | 0.3 | 0.5 |
| FCR | 0.10 | 0.1 | 0.2 | 0.2 | 0.3 |
| FDS | 0.12 | 0.1 | 0.2 | 0.3 | 0.4 |
| FDP | 0.12 | 0.1 | 0.2 | 0.3 | 0.4 |
| ECRB | 0.13 | 0.1 | 0.2 | 0.3 | 0.4 |
| Subscapularis | 1.02 | 0.8 | 1.5 | 2.3 | 3.4 |
| Teres Minor | 0.16 | 0.1 | 0.2 | 0.4 | 0.5 |
| Infraspinatus | 0.76 | 0.6 | 1.1 | 1.7 | 2.6 |
| Supraspinatus | 0.31 | 0.2 | 0.5 | 0.7 | 1.0 |

Table 2 provides a multiplication factor by which the appropriate dosage for other muscles may be determined. For example, the soleus muscle has a mass approximately 2.63 times greater than the lateral gastrocnemius. Where 75U of toxin is efficacious for relaxation of the lateral gastrocnemius, and approximately 0.8 ml of a 100 U/ml concentration of toxin is administered in a patient with a body weight of up to 5 kg, approximately 2.0 ml (i.e., 2.63 times 0.8 ml) is efficacious for relaxation of the soleus. Note that as body weight doubles to 10 kg, 20 kg, and 40 kg, toxin is diluted accordingly but injected in sufficient volume such that the absolute amount of botulinum toxin administered remains the same regardless of muscle size. Increasing muscle mass does not require additional toxin because the number of neuromuscular junctions does not change.

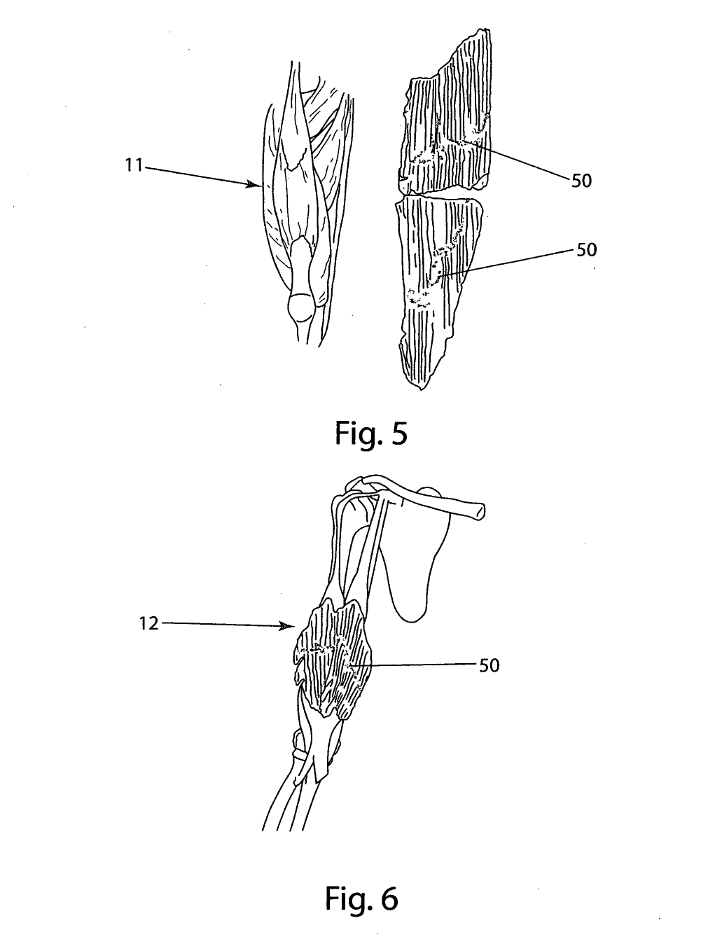

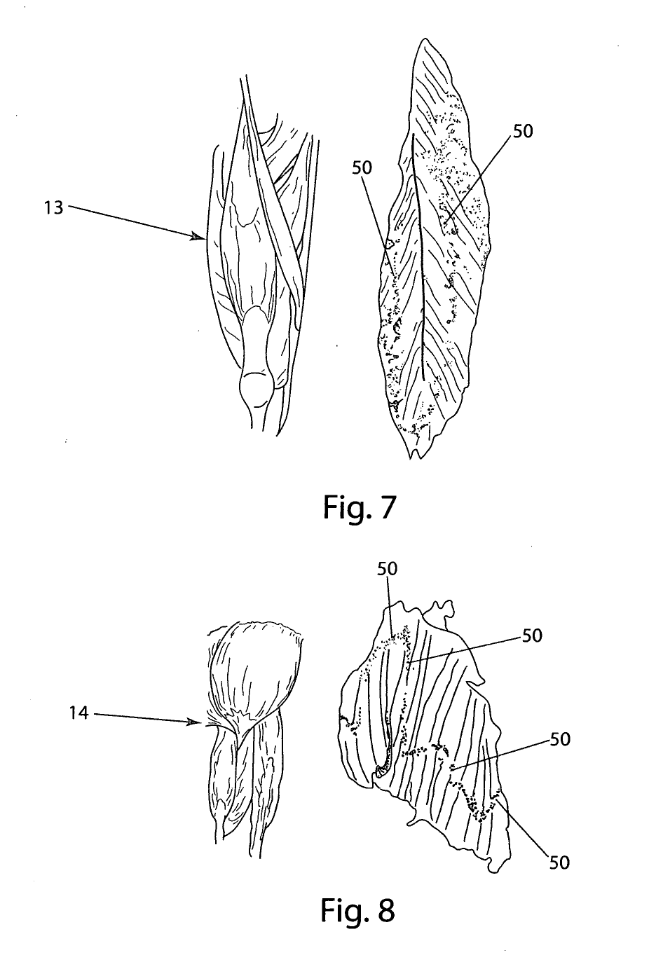

FIG. 4 shows a unipennate muscle 10 with a single transverse band of neuromuscular junctions 50. Intramuscular injection of toxin is most efficacious when delivered within 3.0 cm of this band. Similarly FIG. 5 shows unipennate gracilis muscle 11 with two transverse bands of neuromuscular junctions 50. FIG. 6 shows a bipennate converging biceps brachii muscle 12 having neuromuscular junctions 50 located in an inverted “U” shape. FIG. 7 shows a rectus femoris muscle 13 with two bands of neuromuscular junctions 50 running along its length, and FIG. 8 shows a deltoid muscle 14 with an irregular pattern of neuromuscular junctions 50.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

1. A method for dosing botulinum toxin in a human target muscle to effect a desired treatment while limiting toxin diffusion to non-targeted tissues comprising the steps of:
a) determining the mass of a target muscle;
b) determining the distribution pattern and location of neuromuscular junctions (NMJ) in said target muscle; and,
c) delivering doses of a botulinum toxin A formulation with a charged backbone by intramuscular injection around said distribution pattern and location of said target muscle neuromuscular junctions within a range of about 0.1 cm to about 3.0 cm of said target muscle neuromuscular junctions distribution pattern in an amount corresponding to the quantity of said neuromuscular junctions in said target muscle and the mass of said target muscle.

2. (canceled)

3. The method of claim 1 wherein said charged backbone is selected from the group consisting of a cationic peptide, a hydrocarbon backbone, and a hydrocarbon backbone interrupted by heteroatoms further selected from the group consisting of nitrogen, oxygen, sulphur, silicon, and phosphorous.

4. The method of claim 1 wherein said charged backbone is a polymer comprised of repeating units of amino acids, poly(ethyleneoxy), poly(propyleneamine), polyalkyleneimine, or a heteropolymer.

5. The method of claim 1 wherein said charged backbone is a polypeptide having protein transduction domains.

6. (canceled)

7. The method of claim 1 wherein said target muscle is a unipennate muscle taken from the group consisting of the opponens pollicis, semitendinosus, and brachioradialis, and said neuromuscular junctions are distributed about a transverse midline of said target muscle.

8. The method of claim 1 wherein said target muscle is a unipennate gracilis muscle and said neuromuscular junctions are distributed about two transverse lines on said target muscle.

9. The method of claim 1 wherein said target muscle is a bipennate converging muscle taken from the group consisting of the gastrocnemius and biceps brachii, and said neuromuscular junctions are distributed in a substantially inverted U-shaped pattern on said target muscle.

10. The method of claim 1 wherein said target muscle is a soleus muscle and said neuromuscular junctions are distributed along the length of the muscle fibers.

11. The method of claim 1 wherein said target muscle is a rectus femoris and said neuromuscular junctions are distributed about two longitudinal lines running along the length of said target muscle.

12. The method of claim 1 wherein said target muscle is a deltoid muscle and said neuromuscular junctions are distributed irregularly on said target muscle.

13. (canceled)

14. The method of claim 1 wherein said charged backbone is a polypeptide having protein transduction domains.

15. A method for dosing botulinum toxin in a human muscle comprising the steps of:
a) determining the mass of a target muscle taken from a group consisting of the gastrocnemius and biceps brachii muscles relative to the mass of a lateral gastrocnemius muscle;
b) determining a U-shaped pattern location of neuromuscular junctions (NMJ) in said target muscle; and,
c) injecting a dosage comprising a plurality of doses of a diffusion minimizing botulinum toxin formulation derived from botulinum A toxin having non-native, non-covalently associated molecules including a positively charged backbone, said non-native, non-covalently associated molecules being added in an amount ranging from about 0.1 pg to about 1.0 mg per unit of the botulinum toxin composition of said formulation in an amount that maximizes penetration rate right before saturation and relative to the quantity and location pattern of said neuromuscular junctions in said target muscle, said quantity of neuromuscular junctions being a function of the ratio the mass of said lateral gastrocnemius muscle relative to the mass of said target muscle, the number of doses being additionally mediated by the length and width of the target muscle.

16. The method of claim 15 wherein said charged backbone is selected from the group consisting of a cationic peptide, a hydrocarbon backbone, and a hydrocarbon backbone interrupted by heteroatoms selected from the group consisting of nitrogen, oxygen, sulphur, silicon, and phosphorous.

17. (canceled)

18. The method of claim 15 wherein said positively charged backbone is a polypeptide having protein transduction domains.

19. A method for dosing a botulinum toxin A derived diffusion minimizing botulinum toxin formulation in a human target muscle so that toxin distribution to non-targeted tissue is limited comprising the steps of:
a) determining the mass of a target gastrocnemius muscle relative to the mass of a lateral gastrocnemius muscle;
b) diluting a solution containing said botulinum toxin A diffusion minimizing botulinum toxin formulation so that dilution of the toxin increases in an amount proportionate with body weight;
c) determining the distribution pattern and location of neuromuscular junctions (NMJ) in said target gastrocnemius muscle; and,
d) injecting doses of said diffusion minimizing botulinum toxin formulation including a non-native molecule having a charged backbone, comprising a cationic peptide adjacent said neuromuscular junctions relative to the quantity of said neuromuscular junctions in said target muscle and relative to the ratio of the mass of a lateral gastrocnemius muscle and the mass of said target gastrocnemius muscle to obtain maximum treatment.

20. (canceled)

21. (canceled)
